(19) United States
(12) Patent Application Publication
(10) Pub. No.: US 2025/0188090 A1
(43) Pub. Date: Jun. 12, 2025

(54) A METHOD FOR EXTRACTION OF ALKALOID

(71) Applicant: SUN PHARMACEUTICAL INDUSTRIES (AUSTRALIA) PTY LTD, Port Fairy, Victoria (AU)

(72) Inventors: Geoffrey Wayne STEVENS, Victoria (AU); Kathryn Anne MUMFORD, Victoria (AU); Wen LI, Victoria (AU); Yue WU, Victoria (AU); Hiep THUAN LU, Victoria (AU); Jessica Indah VOVERS, Victoria (AU)

(73) Assignee: SUN PHARMACEUTICAL INDUSTRIES (AUSTRALIA) PTY LTD, Port Fairy, Victoria (AU)

(21) Appl. No.: 18/726,938

(22) PCT Filed: Jan. 5, 2023
(86) PCT No.: PCT/AU2023/050002

§ 371 Date: Jul. 5, 2024

(30) Foreign Application Priority Data
Jan. 7, 2022 (AU) 2022900031

(51) Int. Cl. C07D 489/02

(52) U.S. Cl. CPC C07D 489/02

(57) ABSTRACT

The present invention provides a liquid-liquid extraction process for the extraction of alkaloids from a source, using an alkyl phosphine oxide of Formula A (as described herein) as an extractant in combination with a diluent selected from xylene or limonene; wherein the method comprises the step of contacting the aqueous solution of alkaloid source with an organic layer consisting of an alkyl phosphine oxide of Formula A as extractant and a diluent selected from xylene or limonene.

This application claims priority from Australian Patent Application 2022900031 filed on 7 Jan. 2022, the contents of which are to be taken as incorporated herein by this reference.

### FIELD OF THE INVENTION

The present invention relates to an improved liquid-liquid extraction process for the extraction of alkaloids. Precisely, the present invention provides an improved liquid-liquid extraction process for alkaloids from natural sources such as plants, using an alkyl phosphine oxide (Formula A) (described herein) as an extractant in combination with a diluent selected from xylene or limonene.

### BACKGROUND OF THE INVENTION

The following discussion of the prior art is intended to present the invention in an appropriate technical context, and allows its significance to be properly appreciated. Unless clearly indicated to the contrary, reference to any prior art in this specification should not be construed as an expressed or implied admission that such art is widely known or forms part of common general knowledge in the field.

Solvent extraction is a separation and purification technology that has been widely used in large scale pharmaceutical, mining and chemical industries. In a natural alkaloids manufacturing process, solvent extraction plays a vital role in obtaining high purity alkaloids, such as morphine, which are produced from natural poppies via a series of separation operations. Due to occupational health and safety, environmental and economic concerns, finding effective extractant and environmentally friendly alternatives for traditional volatile organic solvents is attracting more interest.

Solvent extraction is a common chemical processing unit operation used to separate and purify substances, which are difficult to separate using traditional distillation operations. This includes temperature-sensitive pharmaceuticals, azeotropes and leaching solutions containing multiple metal ions. Solvent extraction is based upon the enrichment of a target solute within one of two immiscible liquid phases (aqueous and organic phase). Volatile organic compounds (VOCs), which are derived from petrochemicals such as hydrocarbons (aliphatic, aromatic or halogenated), ethers and aldehydes are primarily used as the organic phase. In addition, VOCs are associated with risks to human health and to the environment due to their toxicity and potential for air pollution. Green solvents have been gaining momentum to replace traditional volatile organic compounds (VOCs) in solvent extraction processes due to environmental and health concerns.

There are a number of prior art documents including journal articles as well as patent documents that describe solvent extraction method such as Separation and Purification Technology 103, p 28-35 (2013); Environmental Science and Pollution Research 27 (31), p 39068-76 (2020); CN109731368A, Analytica Chimica Acta 248 (2), p 501-6 (1991); Journal of Physical and Chemical Reference Data 20 (4), p 713-56 (1991).

There is still a need to develop an industrially viable commercial process for the extraction of alkaloids from the source; which is simple, efficient and cost-effective and yet provides the desired compounds in improved yield and purity. There is also a need to develop a process to extract alkaloids from the source, which is high yielding, efficient and easily scalable.

Inventors of the present invention have developed an improved extraction method which does not involve use of any toxic, hazardous and/or costly solvents. Moreover, the process does not require additional purification steps and critical workup procedure. Accordingly, the present invention provides a liquid-liquid extraction method for extraction of alkaloids; which is simple, efficient, cost effective, environmentally friendly and commercially scalable for large scale operations.

### SUMMARY OF THE INVENTION

In an aspect, the present invention provides a process for the extraction of an alkaloid from the source. Accordingly, the process comprises use of alkyl phosphine oxide or a mixture of alkyl phosphine oxides for the extraction of alkaloid from the source.

In one aspect, the present invention relates to a liquid-liquid extraction process for the extraction of alkaloids from the source, using an alkyl phosphine oxide.

In another aspect, the present invention relates to a liquid-liquid extraction process for the extraction of alkaloids from the source, using a mixture of alkyl phosphine oxides.

According to any of the preceding aspects, an alkyl phosphine oxide is a compound of Formula A:

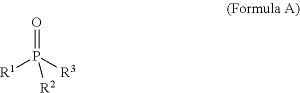

wherein R¹, R² and R³ are each independently selected from C₁-C₁₀ alkyl which is straight chain, branched or cyclic alkyl.

In an aspect, the present invention relates to a liquid-liquid extraction process for the extraction of alkaloids from the source, using an alkyl phosphine oxide as an extractant in combination with a diluent selected from xylene or limonene; wherein the alkyl phosphine oxide is represented by Formula A as,

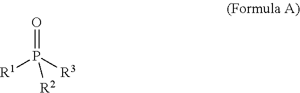

wherein R¹, R² and R³ are each independently selected from C₁-C₁₀ alkyl which is straight chain, branched or cyclic alkyl.

In another aspect, the present invention relates to a liquid-liquid extraction process for the extraction of alkaloid from the source, using an alkyl phosphine oxide or a mixture of alkyl phosphine oxides as an extractant in combination with a diluent selected from xylene or limonene; wherein the alkyl phosphine oxide is represented by Formula A as,

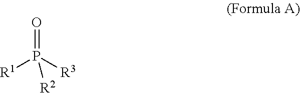

wherein R¹, R² and R³ are each independently selected from C₁-C₁₀ alkyl which is straight chain, branched or cyclic alkyl.

In another aspect, the present invention provides a liquid-liquid extraction process for the extraction of alkaloids from a source using an alkyl phosphine oxide or a mixture of alkyl phosphine oxide of Formula A (described herein) as an extractant and a diluent selected from xylene or limonene.

In another aspect, the present invention relates to a liquid-liquid extraction process for the extraction of alkaloid from a source, comprising the step of contacting the aqueous solution of alkaloid source with an organic layer consisting of an alkyl phosphine oxide of Formula A (described herein) as an extractant and a diluent selected from xylene or limonene.

In another aspect, the present invention provides a liquid-liquid extraction process for the extraction of alkaloids from a source comprising the steps of;
(a) preparing an aqueous phase solution of alkaloids by mixing source with water;
(b) separately, preparing an organic phase comprising one or more alkyl phosphine oxides;
(c) contacting the aqueous phase solution of step (a) with the organic phase of step (b);
(d) separating the organic phase comprising extracted alkaloids.

In another aspect, the present invention provides a liquid-liquid extraction process for the extraction of alkaloids from a source comprises the steps of;
(a) preparing an aqueous phase solution of alkaloids by mixing source with water;
(b) separately, preparing an organic phase consisting of an alkyl phosphine oxide as extractant and a diluent selected from xylene or limonene;
(c) contacting the aqueous solution of step (a) containing alkaloids with an organic layer consisting of an alkyl phosphine oxide as extractant and a diluent selected from xylene or limonene of step (b) having molar concentration of extractant from 0.2M to 0.6M;
(d) maintaining the pH of reaction step (c) ranging from 8 to 10 and ratio of aqueous phase to organic phase (A/O ratio)≤1;
(e) separating the organic phase and subsequent back extraction to provide alkaloids.

In another aspect, the present invention provides a liquid-liquid extraction process for the extraction of alkaloids from a source using one or more alkyl phosphine oxide of Formula A as an extractant in combination with a diluent selected from xylene or limonene in an extraction column, wherein the said process comprises the steps of,
(i) Installing an organic phase distributor at the bottom of the column, consisting an outlet port of organic phase at the top of the column, and installing an aqueous phase distributor at the top of the column, consisting an outlet port of aqueous phase at the bottom of the column,
(ii) disbursing the aqueous phase and organic phase into the column, whereby the two phases undergo extraction and mass transfer using a stack of reciprocating plates inside the column, at pH ranging from 8 to 10, and
(iii) separating the organic phase and subsequent back extraction to provide alkaloids.

In a further aspect, the present invention provides an alkaloid or alkaloids obtained by the liquid-liquid extraction methods and processes described herein. Preferably, the alkaloid or alkaloids are selected from morphine, codeine, oripavine, thebaine or noscapine.

In an aspect, the present invention relates to a process of extraction of morphine from the source.

In another aspect, the present invention relates to a process of extraction of oripavine from the source.

In yet another aspect, the present invention relates to a process of extraction of morphine or oripavine from the source, wherein the process comprises use of alkyl phosphine oxide as an extractant and wherein the alkyl phosphine oxide is selected from the group consisting of dioctyl-monohexyl phosphine oxide, monooctyl-dihexyl phosphine oxide, trioctyl phosphine oxide and trihexyl phosphine oxide, or a mixture thereof.

### BRIEF DESCRIPTION OF THE DRAWINGS

FIG. 1 depicts the extraction columns setup.

FIG. 2 depicts the isotherm of rich extract morphine using 0.2 M alkyl phosphine oxide (A) in xylene. Isotherm b is an expansion of the shaded area of isotherm a.

FIG. 3 depicts the Morphine isotherms using different extractant mixtures.

### DETAILED DESCRIPTION OF THE INVENTION

Various embodiments and variants of the present invention are described hereinafter.

The term “liquid-liquid extraction,” as used herein, refers to a general method of extraction which comprises mass transfer from one liquid phase to another liquid phase by physical contact of the two liquid phases, for example mass transfer from aqueous phase to the organic phase.

The term “extractant” as used herein, refers to a liquid which is capable of extraction or separating out desired substance by extraction when it is mixed with others liquid phase or physically contacted with other liquid solution such as alkaloid rich aqueous phase solution.

In an embodiment, the extractant is selected from at least one or mixture of more than one alkyl phosphine oxides of Formula A:

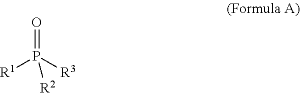

wherein R¹, R² and R³ are each independently selected from C₁-C₁₀ alkyl which is straight chain, branched or cyclic alkyl.

In an embodiment, R¹, R² and R³ of the alkyl phosphine oxide of Formula A are independently selected from hexyl or octyl.

In another embodiment, the alkyl phosphine oxide of Formula A, is selected form the group consisting of dioctyl-monohexyl phosphine oxide, monooctyl-dihexyl phosphine oxide, trioctyl phosphine oxide and trihexyl phosphine oxide, or a mixture thereof.

In a preferred embodiment, the alkyl phosphine oxide of Formula A is used as an extractant, which is a composite mixture comprising of dioctyl-monohexyl phosphine oxide (10-22%), monooctyl-dihexyl phosphine oxide (10-16%), trioctyl phosphine oxide (5-8%) and trihexyl phosphine oxide (5-8%). Advantageously, the alkyl phosphine oxide mixture is liquid at room temperature.

The term “back extraction” as used herein, refers to a process involving recovering the extraction product from extractant phase. For instance, the alkaloid rich extractant phase require to undergo back extraction method in order to recover the extracted alkaloid such as morphine.

The phrases “distribution coefficient (D)” and “partition coefficient (PC)” are used interchangeably and mean a ratio of concentration of solute in two different immiscible solvents at equilibrium. For the purpose of present invention D or PC is a ratio of concentration of alkaloid in the organic phase to that of concentration in aqueous phase. In particular the organic phase comprises compound of Formula A in xylene or limonene.

According to an embodiment, the present invention provides a process for extraction of an alkaloid from the source.

In an embodiment, the process comprises use of alkyl phosphine oxide for extraction of alkaloid from the source. In another embodiment, the process comprises use of mixture of alkyl phosphine oxides for the extraction of alkaloid from the source.

According to a specific embodiment, the present invention relates to a liquid-liquid extraction process for the extraction of alkaloids from the source, using an alkyl phosphine oxide.

According to another specific embodiment, the present invention relates to a liquid-liquid extraction process for the extraction of alkaloids from the source, using a mixture of alkyl phosphine oxides.

In an embodiment, the alkyl phosphine oxide is of Formula A. In an embodiment, the alkyl phosphine oxide of Formula A is diluted with a diluent. The diluent may be selected from xylene or limonene.

According to an embodiment, the concentration of the alkyl phosphine oxide of Formula A in xylene or limonene is from 0.2 M to 0.6 M.

Accordingly, the present invention provides a liquid-liquid extraction process for alkaloids from a source, using an alkyl phosphine oxide as an extractant in combination with a diluent selected from xylene or limonene; wherein the alkyl phosphine oxide is represented by Formula A as,

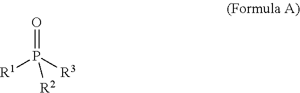

wherein R¹, R² and R³ are each independently selected from C₁-C₁₀ alkyl which is straight chain, branched or cyclic alkyl.

In an embodiment, the source of alkaloid is any biological resources such as plant. In another embodiment, the source of alkaloid is poppy straw from the biological resources such as plants. In yet another embodiment, the source of alkaloid is an extract obtained from milled poppy straw by repeated washings using water to obtain aqueous alkaloid-heavy rich extract. The rich extract of morphine, for example may comprise morphine concentration from 0.3 to 0.4 w/v %.

In an embodiment, the alkyl phosphine oxide of Formula A (described herein) is used as an extractant.

In a preferred embodiment, the alkyl phosphine oxide is represented by Formula A,

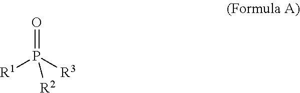

wherein R¹, R² and R³ are each independently selected from C₅-C₁₀ alkyl which is straight chain, branched or cyclic alkyl.

In an embodiment, the R¹, R² and R³ of the alkyl phosphine oxide of Formula A are independently selected from hexyl or octyl.

In a preferred embodiment, the alkyl phosphine oxide of Formula A, is selected form the group consisting of dioctyl-monohexyl phosphine oxide, monooctyl-dihexyl phosphine oxide, trioctyl phosphine oxide and trihexyl phosphine oxide, or a mixture thereof.

In an embodiment, the alkyl phosphine oxide of Formula A is a trialkyl-phosphine oxides or a mixture thereof. More preferably, the alkyl phosphine oxide represented by Formula (A) is a composite mixture of 94% trialkyl-phosphine oxides.

In one embodiment, the alkyl phosphine oxide of Formula A is used as an extractant, which is a composite mixture comprising of dioctyl-monohexyl phosphine oxide (10-22%), monooctyl-dihexyl phosphine oxide (10-16%), trioctyl phosphine oxide (5-8%) and trihexyl phosphine oxide (5-8%). Advantageously, the alkyl phosphine oxide mixture is liquid at room temperature.

In an embodiment, a diluent to combine with the extractant is selected from xylene or limonene.

In a preferred embodiment, the diluent to combine with the extractant is xylene.

In another preferred embodiment, the diluent to combine with the extractant is d-limonene.

In a specific embodiment, the process for extraction of alkaloids from a source comprises the step of contacting an aqueous solution of alkaloid source with an organic layer consisting of an alkyl phosphine oxide of Formula A as an extractant and a diluent selected from xylene or limonene.

According to an embodiment, the present invention provides a liquid-liquid extraction process for the extraction of alkaloids from a source comprising the steps of;
(a) preparing an aqueous phase solution of alkaloids by mixing source with water;
(b) separately, preparing an organic phase comprising one or more alkyl phosphine oxides;
(c) contacting the aqueous phase solution of step (a) with the organic phase of step (b);
(d) separating the organic phase comprising extracted alkaloids.

In a specific embodiment, the process for the extraction of alkaloids from a source comprises the steps of,
(a) preparing an aqueous phase solution of alkaloids by mixing source with water;
(b) separately, preparing an organic phase consisting of an alkyl phosphine oxide as extractant and a diluent selected from xylene or limonene;
(c) contacting the aqueous solution of step (a) containing alkaloids with an organic layer consisting of an alkyl phosphine oxide as extractant and a diluent selected from xylene or limonene of step (b) having molar concentration of extractant from 0.2M to 0.6M;
(d) maintaining the pH of reaction step (c) ranging from 8 to 10 and ratio of aqueous phase to organic phase (A/O ratio)≤1;
(e) separating the organic phase and subsequent back extraction to provide alkaloids.

The aqueous phase solution of step (a) is an alkaloid rich aqueous phase solution, prepared by mixing alkaloid source with water. For instance, the alkaloid rich aqueous phase solution such as morphine rich aqueous phase was prepared from the concentrate of Poppy Straw; wherein milled poppy straw was washed repeatedly with water to obtain the aqueous phase alkaloid-heavy rich extract, which primarily contains morphine with lower levels of other opiate compounds and some organic matter. This aqueous phase solution is dark brown and opaque, and if left over time it was observed that a layer of particulate matter settled out at the bottom of the solution.

The organic phase of step (b) is an organic phase consisting of an extractant and a diluent, wherein the extractant is selected from alkyl phosphine oxide of Formula A and the diluent selected from xylene or limonene.

The alkaloid extraction as of step (c) was achieved by contacting the aqueous solution containing alkaloids with an organic layer consisting of an alkyl phosphine oxide as extractant and a diluent selected from xylene or limonene, wherein the organic phase having molar concentration of extractant from 0.2M to 0.6M.

In a preferred embodiment, the liquid-liquid extraction method of the present invention is performed at the molar concentration of the extractant with respect to the diluent ranging from 0.2M to 0.6M.

The alkaloid extraction as of step (d) is achieved by maintaining the pH of the extraction system ranging from 8 to 10 and also ratio of aqueous phase to organic phase (A/O ratio)≤1.

In an embodiment, the liquid-liquid extraction method is performed at a pH ranging from 8 to 10. In a preferred embodiment, the liquid-liquid extraction method is performed at a pH about 9. The term ‘about’ used in the context of pH 9, represents the pH ranging from 8.95 to 9.05.

In a preferred embodiment, the liquid-liquid extraction method is performed at aqueous phase to organic phase ratio ≤1 (O/A ratio).

The alkaloid extraction as of step (e) wherein the extracted alkaloid is recovered from the organic phase by the back extraction method. The separated alkaloid is ‘isolated’ by any of the general isolation steps comprising separation from organic phase, filtration, decantation, distillation, evaporation of solvent, precipitation, concentration, crystallization, centrifugation, recrystallization, washing and drying.

In a general embodiment, the liquid-liquid extraction method of the present invention for the extraction of alkaloids is used for the extraction of morphine, codeine, oripavine, thebaine or noscapine.

Accordingly, the liquid-liquid extraction method of the present invention shows the morphine extraction efficiency >95% from the aqueous phase of the source.

In an embodiment, the liquid-liquid extraction method of the present invention is extended to the large scale commercial extraction using an extraction columns setup as depicted in FIG.-1.

In an embodiment, the present invention provides an alkaloid or alkaloids obtained by the liquid-liquid extraction methods and processes described herein.

Accordingly, in one specific embodiment, the present invention provides an alkaloid or alkaloids obtained from the source by a liquid-liquid extraction method, using an alkyl phosphine oxide as an extractant in combination a diluent selected from xylene or limonene,
wherein the alkyl phosphine oxide is represented by Formula A as,

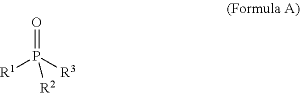

wherein R¹, R² and R³ are each independently selected from C₁-C₁₀ alkyl which is straight chain, branched or cyclic alkyl.

In a preferred embodiment, the alkaloid or alkaloids are selected from morphine, codeine, oripavine, thebaine or noscapine.

In a specific embodiment, the liquid-liquid extraction process for the extraction of alkaloids from a source using an alkyl phosphine oxide of Formula A as an extractant in combination with a diluent selected from xylene or limonene in an extraction column, wherein the said process comprises the steps of,
(i) Installing an organic phase distributor at the bottom of the column, consisting an outlet port of organic phase at the top of the column, and installing an aqueous phase distributor at the top of the column, consisting an outlet port of aqueous phase at the bottom of the column,
(ii) disbursing the aqueous phase and organic phase into the column, whereby the two phases undergo extraction and mass transfer using a stack of reciprocating plates inside the column, at pH ranging from 8 to 10, and
(iii) separating the organic phase and subsequent back extraction to provide alkaloids.

In an embodiment, the liquid-liquid extraction column set up consist of a stack of reciprocating plates which is placed inside the extraction column. The stack of reciprocating plates is made of stainless steel and placed in the centre of the column.

In an embodiment, the stack of plates of the liquid-liquid extraction column set up are reciprocated using a variable speed pump installed at the top of the column, and the reciprocating amplitude is adjusted through a slider crank with a fly wheel which was attached to the pump.

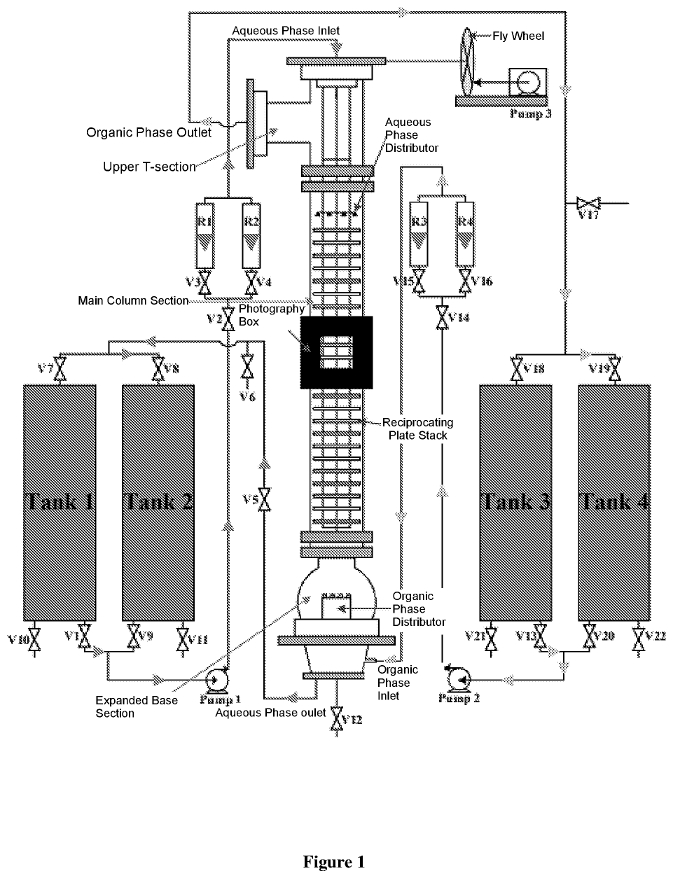

The extraction columns setup as depicted in FIG. 1, wherein an organic phase distributor is installed at the bottom of the column, wherein the outlet port of organic phase is set at the top, and an aqueous phase distributor is equipped at the top of the column, wherein outlet port of aqueous phase is set at the bottom. Both, top and bottom of the column are having expanded areas which are used as buffer zones for the organic and aqueous phases, respectively. A stack of perforated plates made of stainless steel are filled in the centre of the column, which were reciprocated using a variable speed pump installed at the top of the column. The reciprocating amplitude was adjusted through a slider crank with a fly wheel which was attached to the pump.

As is shown in FIG. 1, the Tank 1 and Tank 2 are used for the aqueous phase, while Tank 3 and Tank 4 for the organic phase. Before starting the extraction, Tank 1 was loaded with the aqueous phase solution, and Tank 3 was filled with 0.2 M trialkylphosphine oxide in xylene. The pH of the aqueous phase was adjusted to about 9 using the appropriate base or acid solution, such as NaOH or H₂SO₄ solutions. The aqueous phase was pumped from Tank 1 to the column, while the organic phase was pumped from Tank 3 to the bottom of the column. Two phases underwent extraction and mass transfer with the assistance of the reciprocating plates inside the column. Empty Tank 2 and Tank 4 were used to collect the raffinate and alkaloid rich solvent, respectively.

The inventors of the present invention performed mass transfer study using morphine solutions which was prepared from upstream alkaloid-heavy rich extract. Sulfuric acid (98 wt %) and sodium hydroxide pellets (90%) were used to prepare different concentrations of acid and base to adjust the pH of morphine solution. The optimised pH value for morphine extraction was found about 9.

The trialkylphosphine oxide was pre-washed using 0.5 M sulfuric acid to remove any metal impurities, and then 0.2 M trialkylphosphine oxide in xylene solution was prepared. The prepared solvent was washed again using strong base to remove any residue acids, and to adjust the pH to 9. The pH of the morphine solution was also adjusted to 9 using sulfuric acid (H₂SO₄) and sodium hydroxide (NaOH) solution before conducting mass transfer process. Ultrapure water from a Milli-Q purification system (Elix Millipore, AU) was used for morphine equilibrium isotherm tests.

The extraction column which was used to perform morphine mass transfer from the alkaloid-heavy rich extract was having following specification as Table: 1—

TABLE 1

| Column diameter (D_{c}) | 50 | mm |
|---|---|---|
| Effective column height (H_{c}) | 1.6 | m |
| Effective column volume (Vₑ) | 3140 | cm³ |
| Number of plates (Nₚ) | 35 | |
| Plate thickness (e) | 3 | mm |
| Plate Material | Stainless steel | |
| Perforation diameter (dₕ) | 12.7 | mm |
| Hole pitch (Triangular) (P) | 17 | mm |
| Plate spacing (h_{c}) | 47 | mm |
| Plate free area (S) | 0.452 | |
| Amplitude (A) | 15 | mm |

The aqueous phase containing rich extract morphine solution was initially pumped in the empty column via a liquid distributor at the top of the Karr plates. When the distributor was submerged in the aqueous solution, the organic phase started flowing into the column via the distributor located at the bottom. The flowrates of both phases were adjusted to the pre-set values. The reciprocating plates was also turned on and the frequency adjusted to the designed values. The pH of the inlet aqueous phase was measured and controlled at around 9 via an in-situ pH probe.

The resultant organic layer of the preferred experimental conditions, that is the alkyl phosphine oxide of Formula A in xylene (0.2M, 7.91v %) which was previously contacted with morphine rich extract aqueous phase. This rich organic phase, loaded with morphine and trace alkaloids, was contacted with 2 wt % NaOH to back extract out morphine, resulting in a lean organic phase and concentrated extract. The 0.2M alkyl phosphine oxide of Formula A in xylene was the continuous phase, with aqueous NaOH being the dispersed phase. Stainless steel plates were used in the plate stack to reduce changeover time between extraction and stripping experiments, although generally for continuous organic phase a hydrophobic plate surface is preferred for optimal plate wetting, droplet formation and performance.

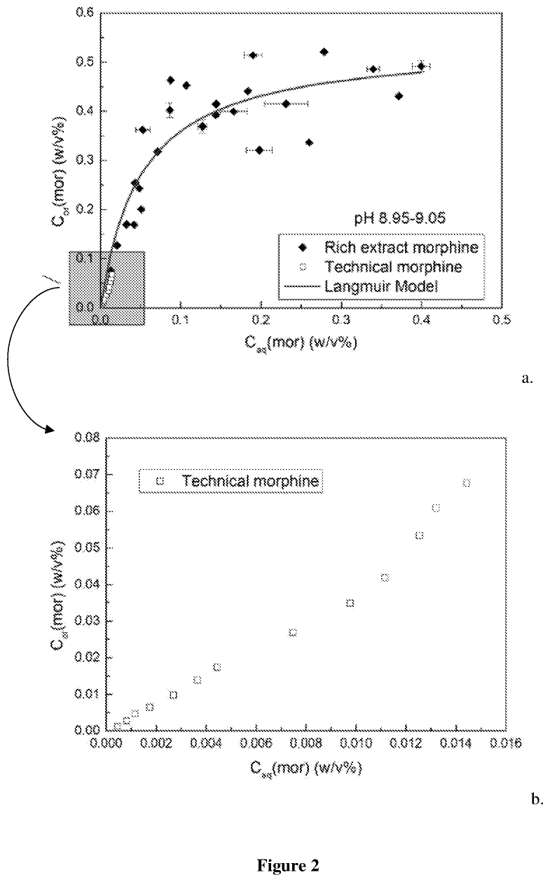

FIG. 2 illustrates that in rich extract morphine solution, the morphine concentration at pH 9 was about 0.3 to 0.4 w/v %, which is much higher than that of technical morphine (0.0208 w/v %). This possibly due to the impurities existing in the rich extract solution, acting as surfactants to increase the solubility of morphine. The equilibrium isotherm tests using the rich extract morphine solution and 0.2 M alkyl phosphine oxide of Formula A/xylene and its corresponding the Langmuir model are given in Error! Reference source not found. The FIG. 2 illustrates the significant difference made by acid dilution of aqueous phase samples prior to UPLC analysis.

The concentration of morphine in aqueous phase increases, so does the concentration of morphine in organic phase. The saturated concentration of morphine in the organic phase (0.2M alkyl phosphine oxide of Formula A in xylene) was around 0.45 w/v %. However, the sample preparation of the equilibrated aqueous phase made a significant difference in the concentration of morphine detected by UPLC. It is observed during study of mass transfer of rich extract morphine solution by extraction columns using 0.2 M alkyl phosphine oxide of Formula A in xylene that at operating conditions investigation, the mass transfer efficiency of rich extract morphine solution was up to 95%. In addition, the equilibrium isotherm of morphine using 0.2 M alkyl phosphine oxide of Formula A/xylene at pH 9 was measured, and the correlation was determined using the Langmuir model, as FIG. 2.

The morphine extraction efficiency and isotherm analysis was determined by the method wherein the alkyl phosphine oxides of Formula A was selected as an extractant, and xylene or d-limonene was selected as diluent. The representative extractant solution concentration selected from 0.2 M (7.9 v/v %) and 0.6 M (23.7 v/v %) of alkyl phosphine oxide of Formula A are prepared using d-limonene, and 0.2 M and 0.6 M solutions of alkyl phosphine oxide of Formula A are also prepared using xylene for comparison. These four extractant mixtures are used to determine morphine extraction efficiency and to measure morphine isotherms.

Table 2 summarizes the morphine extraction efficiency achieved by the liquid-liquid extraction method of the present invention. The extraction efficiency of the morphine was demonstrated representatively using the same 4 different solvent systems, as selected from 0.2 M (7.9 v/v %) and 0.6 M (23.7 v/v %) of alkyl phosphine oxide of Formula A are prepared using xylene, and 0.2 M and 0.6 M of alkyl phosphine oxide of Formula A are also prepared using d-limonene for comparison. Accordingly, it is evident from the Table 2 that upto 92% single stage morphine extraction efficiency was achieved using 0.6M (23.7% v/v) solution of alkyl phosphine oxide of Formula A in xylene, wherein the saturated concentration of morphine achievable in the organic phase was about 0.88% w/v. The pH was optimized at about 9, at which highest extraction efficiency was observed. Additionally, upto 66% single stage morphine extraction efficiency was observed at 0.2M (7.91% v/v) solution of alkyl phosphine oxide of Formula A in xylene, wherein the saturated concentration of morphine achievable in the organic phase was about 0.45% w/v.

TABLE 2
morphine extraction efficiency experimentation

| | | | | | Single stage |
|---|---|---|---|---|---|
| | | | saturated concentration | | morphine |
| Extraction solvent | | Molar | of morphine achievable | | extraction |
| system | pH | concentration | in the organic phase | | efficiency |
| alkyl phosphine oxide | ~9 | 0.2M | 0.45% | w/v | 66% |
| (Formula A) in xylene | (8.95-9.05) | (7.91% v/v) | | | |
| alkyl phosphine oxide | ~9 | 0.6M | 0.88% | w/v | 92% |
| (Formula A) in xylene | (8.95-9.05) | (23.7% v/v) | | | |
| alkyl phosphine oxide | ~9 | 0.2M | 0.24% | w/v | 83% |
| (Formula A) in d-limonene | (8.95-9.05) | (7.91% v/v) | | | |
| alkyl phosphine oxide | ~9 | 0.6M | 0.56 to 0.61% | w/v | 86% |

| | | |
|---|---|---|
| (Formula A) in d-limonene | (8.95-9.05) | (23.7% v/v) |

Alternately, it is also evident from Table 2 that upto 86% single stage morphine extraction efficiency was observed at 0.6M (23.7% v/v) solution of alkyl phosphine oxide of Formula A in d-limonene, wherein the saturated concentration of morphine achievable in the organic phase was about 0.56 to 0.61% w/v. In comparison, upto 83% single stage morphine extraction efficiency was observed at 0.2M (7.91% v/v) solution of alkyl phosphine oxide of Formula A in d-limonene, wherein the saturated concentration of morphine achievable in the organic phase was about 0.24% w/v. The comparison study was further extended with known extraction systems such as amyl alcohol in xylene, wherein ratio aqueous phase with organic phase (A/O ratio) was one of the contributing factor. Accordingly, as depicted in Table 2a that over 95% of multistage morphine extraction efficiency was observed at 0.2M (7.91% v/v) solution of alkyl phosphine oxide of Formula A in xylene, wherein the saturated concentration of morphine achievable in the organic phase was about 0.45% w/v. In this process, the ratio aqueous phase with organic phase (A/O ratio) was ˜ 0.6 with continuous phase velocity 0.0025 m/s˜ 0.3 L/min and agitation rate ≥ 0.027 m/s. The Table 2a also depicts that the about 97.8% of multistage morphine extraction efficiency was observed at 4.6M (50% v/v) solution of amyl alcohol in xylene, wherein the saturated concentration of morphine achievable in the organic phase was about 0.16% w/v. In this process, the ratio aqueous phase with organic phase (A/O ratio) was ˜ 0.5 with continuous phase velocity 0.0066 m/s ˜63 L/min. It is comparative that the extractant containing amyl alcohol in xylene require to be used in higher molar concentration to achieve equivalent multistage morphine extraction efficiency as with alkyl phosphine oxide of Formula A in xylene.

TABLE 2a
Morphine extraction efficiency experimentation comparison

| | | | saturated | | | | |
|---|---|---|---|---|---|---|---|
| | | | concentration | | | | |
| | | | of morphine | Multistage | | | |
| Extraction | | | achievable in | morphine | A/O ratio | continuous | |
| solvent | | Molar | the organic | extraction | (aqueous/ | phase | agitation |
| system | pH | concentration | phase | efficiency | organic) | velocity | rate |
| alkyl | ~9 | 0.2M | 0.45% w/v | over 95% | ≤0.68 | 0.0025 m/s | ≥0.027 |
| phosphine | (8.95-9.05) | (7.91% v/v) | | | | ~0.3 L/min | m/s |
| oxide | | | | | | | |
| (Formula A) | | | | | | | |
| in xylene | | | | | | | |
| amyl alcohol | ~9 | 4.6M | 0.16% w/v | 97.8% | 0.5 | 0.0066 m/s | N/A - |
| in xylene | (8.95-9.05) | (50% v/v) | | | | ~63 L/min | Packed |
| | | | | | | | column |

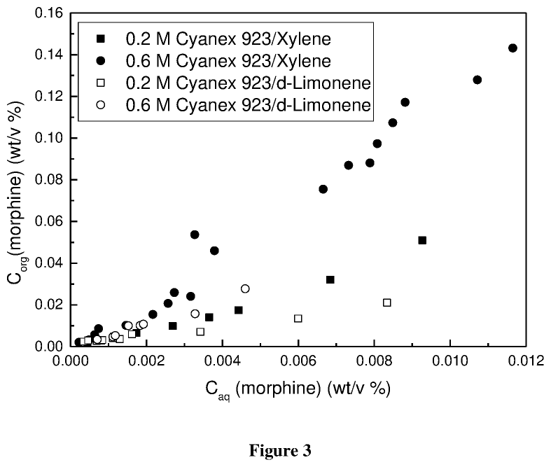

The morphine isotherms measurement study was also done using the same 4 different solvent systems, as selected from 0.2 M (7.9 v/v %) and 0.6 M (23.7 v/v %) of alkyl phosphine oxide of Formula A are prepared using d-limonene, and 0.2 M and 0.6 M of alkyl phosphine oxide of Formula A are also prepared using xylene for comparison. The pH was optimised at about 9. To calculate the concentration in the organic phase, back extraction of organic phase using 0.5 M H₂SO₄ after equilibrium was required. Both aqueous phase and back extraction aqueous phase were measured using a UPLC to determine the morphine concentration in the aqueous phase and organic phase after equilibrium. Accordingly, FIG. 3 depicts the Morphine isotherms using different extractant mixtures and the partition coefficients are given in Table 3.

TABLE 3
Partition coefficients of morphine isotherm at pH 9

| Solvent | alkyl phosphine oxide | Partition |
|---|---|---|
| system | concentration (M) | Coefficient (—) |
| Xylene | 0.2 | 5.41 |
| Xylene | 0.6 | 12.7 |
| d-limonene | 0.2 | 2.21 |
| d-limonene | 0.6 | 5.97 |

Accordingly, Table 3 indicates that the extraction solvent system consisting of alkyl phosphine oxide of Formula A in xylene performs better than extraction solvent system alkyl phosphine oxide of Formula A in d-limonene. It is further evident that, 0.2 M alkyl phosphine oxide of Formula A in xylene has comparable performance with 0.6 M alkyl phosphine oxide of Formula A in d-limonene, which means the quantity of extractant used with xylene can be 3 times less than that used in d-limonene. A possible reason for this is that an emulsion was observed and a third stable phase was formed during the isotherm tests, leading to a significant loss of the alkaloid in the case of the 0.6M alkyl phosphine oxide in d-limonene.

Similarly the extraction efficiency of the compound of Formula A was studied for other alkaloids such as oripavine and codeine.

For oripavine the extraction efficiency of 0.2 M compound of Formula A in xylene was highest at a pH of about 8.9 and of 0.6 M compound of Formula A in xylene was highest at a pH of about 8.7.

The maximum distribution coefficient (D) value for oripavine in 0.2 M compound of Formula A in xylene was 24.8, which corresponds to 96% in the solvent after a 1-stage extraction and a theoretical number of stages of 1.03 for full extraction. For 0.6 M the maximum D value was 46.0, which also corresponds to 98% in the solvent after a 1-stage extraction. An estimated D value for the extraction process using 15% w/v of amyl alcohol in xylene is ˜6, with a 1-stage efficiency of 85%. Thus the performance of compound of Formula A in xylene is superior to the process involving amyl alcohol in xylene for extracting oripavine. The surprisingly higher values of D for oripavine in the compound of Formula A in xylene as compared to amyl alcohol (currently known process involving extraction of oripavine) suggests the higher efficiency of compound of Formula A in the extraction process. It would be appreciated by the skilled person that the theoretic number of stages is calculated using following formula:

$$Ns = \frac{log ( \frac{1 - E}{Xn} ) + E}{log ( \frac{1}{E} )}$$

Where,
Ns=theoretical number of stages
Xn=unextracted fraction, and E=extraction factor
wherein,

$$Xn = \frac{Average conc in the aqueous}{Average conc in the aqueous + average conc in the organic}$$

$$E = \frac{A q ueous volume}{Organic volume \times partition coefficient}$$

Similarly for codeine, the extraction efficiency of compound of Formula A in xylene with both 0.2 M and 0.6 M was highest at a pH between 9 and 10. The maximum D values for codeine were about 4.4 for 0.2 M compound of Formula A in xylene and 7.1 for 0.6 M compound of Formula A in xylene.

On the other hand in 0.2 M compound of Formula A in limonene, the optimum pH value for extraction of oripavine was found to be about 9 to 9.5. The maximum D value of oripavine using 0.2 M compound of Formula A in limonene was 14.5, which corresponds to 93.3% in the solvent after a 1-stage extraction and a theoretical number of stages of 1.04.

In a particular embodiment, the present invention relates to a process of extraction of morphine from the source.

In another particular embodiment, the present invention relates to a process of extraction of oripavine from the source.

In an embodiment, the present invention relates to a process of extraction of morphine or oripavine from the source, wherein the process comprises use of alkyl phosphine oxide as an extractant and wherein the alkyl phosphine oxide is selected from the group consisting of dioctyl-monohexyl phosphine oxide, monooctyl-dihexyl phosphine oxide, trioctyl phosphine oxide and trihexyl phosphine oxide, or a mixture thereof.

According to a particular embodiment, the present invention relates to a process of extraction of morphine or oripavine from the source, wherein the process comprises use of alkyl phosphine oxide as an extractant, and wherein the alkyl phosphine oxide is selected from the group consisting of dioctyl-monohexyl phosphine oxide, monooctyl-dihexyl phosphine oxide, trioctyl phosphine oxide and trihexyl phosphine oxide, or a mixture thereof, wherein the alkyl phosphine oxide is diluted using a diluent selected from the group of xylene or d-limonene and wherein the concentration of the alkyl phosphine oxide in the diluent is from 0.2M to 0.6 M.

Inventors of the present invention, during experimentation observed that a significant pH drop occurred after contacting aqueous solutions with solvent systems containing alkyl phosphine oxides of Formula A. This therefore required multiple iterations of pH adjustment and contacting to reach equilibrium, until the target pH range was reached (pH 8.95-9.05). As mentioned earlier, the alkyl phosphine oxides is a mixture of trialkyl phosphine oxides and immiscible in water (0.01 g/L solubility in water at 25° C.) so the extractant itself is unable to release H+ or OH and alter the pH of aqueous solutions. However, after contacting alkyl phosphine oxides with RO water (Aqueous/Organic ratio (A/O)=2), the pH of the RO water phase dropped from 7.00 to 2.72. Similar decreases in pH were observed when mixing 0.2M alkyl phosphine oxides in xylene or limonene with RO water.

Significantly, the process of the present invention is useful for extraction of pure morphine, which is used for synthesis of codeine and then subsequently codeine phosphate. Also the pure morphine obtained from the instantly designed extraction method as further converted to pholcodine as well as oxymorphone.

Advantageously, the extraction solvent system used in the present invention containing alkyl phosphine oxide of Formula A as extractant and other diluent, is considered as green solvent alternatives for the traditionally used petroleum based solvents.

In an embodiment, the alkaloid or alkaloids obtained by the extraction process of present invention are subsequently converted to a therapeutically effective compound or derivatives thereof.

In another embodiment, the alkaloid or alkaloids obtained by the extraction process of present invention are subsequently converted to a therapeutically effective compounds or derivatives thereof, such as, but not limited to naltrexone, nalbuphine and buprenorphine.

In one aspect, the present invention relates to a liquid-liquid extraction process for the extraction of alkaloids from the source, using an alkyl phosphine oxide as an extractant in combination with a diluent selected from xylene or limonene;
wherein the alkyl phosphine oxide is represented by Formula A as,

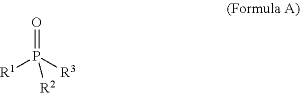

wherein R¹, R² and R³ are each independently selected from C₁-C₁₀ alkyl which is straight chain, branched or cyclic alkyl;
wherein the alkaloids are subsequently converted to a therapeutically effective compound or derivatives thereof.

In an embodiment, the alkaloid or alkaloids obtained by the extraction process of present invention or the subsequently obtained therapeutically effective compounds and derivatives thereof; are used for the preparation of a medicament.

In an embodiment, the alkaloid or alkaloids obtained by the extraction process of present invention or the subsequently obtained therapeutically effective compounds and derivatives thereof, such as, but not limited to naltrexone, nalbuphine and buprenorphine; are used for the preparation of a medicament.

The invention is further illustrated by the following examples which are provided to be exemplary of the invention, and do not limit the scope of the invention. While the present invention has been described in terms of its specific embodiments, certain modifications and equivalents will be apparent to those skilled in the art and are intended to be included within the scope of the present invention.

### EXAMPLES

Extractant: The alkyl phosphine oxide of Formula A was used as extractant, which is a composite mixture comprising of dioctyl-monohexyl phosphine oxide (10-22%), monooctyl-dihexyl phosphine oxide (10-16%), trioctyl phosphine oxide (5-8%) and trihexyl phosphine oxide (5-8%).

### Example 1: Extraction of morphine using 0.2 M alkyl phosphine oxide of Formula A in xylene

### Preparation of Alkyl Phosphine Oxide of Formula A: Xylene Solvent System

The alkyl phosphine oxide (7.91 ml) was diluted to volume in a 100 ml volumetric flask with plant xylene, the mixture was inverted 20 times to combine the liquids. The prepared solvent was then washed with 0.1M sulphuric acid solution at a 50 ml to 50 ml ratio and allowed to settle for 15 min, the heavy phase was removed and discarded.

Extraction of Morphine from the Aqueous Phase into the Organic Phase

Rich extract (20 ml, ˜0.3-0.4% w/v) was adjusted from ˜12 via addition of 5M sulphuric acid dropwise with mixing until pH 9.0 was achieved. This aqueous phase was then combined with 20 ml of the washed organic phase and mixed on a vertical orbital (Ratek suspension mixer) for 60 minutes. The biphasic system was allowed to separate and each phase was assayed. The single stage extraction efficiency for morphine was determined as 66%.

### Example 2: Extraction of Morphine Using 0.6 M Alkyl Phosphine Oxide of Formula A in Xylene

### Preparation of Alkyl Phosphine Oxide of Formula A: Xylene Solvent System

The alkyl phosphine oxide (47.46 ml) was diluted to volume in a 200 ml volumetric flask with plant xylene, the mixture was inverted 20 times to combine the liquids. The prepared solvent was then washed with 0.1M sulphuric acid solution at a 50 ml to 50 ml ratio of solvent to wash and allowed to settle for 15 min, the heavy phase was removed and discarded.

Extraction of Morphine from the Aqueous Phase into the Organic Phase

Rich extract (20 ml, ˜0.1-0.2% w/v) was adjusted from ˜12 via addition of 5M sulphuric acid dropwise with mixing until pH 9.0 was achieved. This aqueous phase was then combined with 20 ml of the washed organic phase and mixed on a vertical orbital (Ratek suspension mixer) for 60 minutes. The biphasic system was allowed to separate and each phase was assayed. The single stage extraction efficiency for morphine was determined as 92%.

### Example 3: Extraction of Morphine Using 0.2 M Alkyl Phosphine Oxide of Formula A in d-Limonene

Preparation of Alkyl Phosphine Oxide of Formula A: d-Limonene Solvent System

The alkyl phosphine oxide (15.82 ml) was diluted to volume in a 200 ml volumetric flask with d-limonene, the mixture was inverted 20 times to combine the liquids. The prepared solvent was then washed with 0.1M sulphuric acid solution at a 50 ml to 50 ml ratio of solvent to wash and allowed to settle for 15 min, the heavy phase was removed and discarded.

Extraction of Morphine from the Aqueous Phase into the Organic Phase

Rich extract (20 ml, 0.3-0.4% w/v) was adjusted from ˜12 via addition of 5M sulphuric acid dropwise with mixing until pH 9.0 was achieved. This aqueous phase was then combined with 20 ml of the washed organic phase and mixed on a vertical orbital (Ratek suspension mixer) for 60 minutes. The biphasic system was allowed to separate and each phase was assayed. The single stage extraction efficiency for morphine was determined as 83%.

### Example 4: Extraction of Morphine Using 0.6 M Alkyl Phosphine Oxide of Formula A in d-Limonene

Preparation of Alkyl Phosphine Oxide of Formula A: d-Limonene Solvent System

The alkyl phosphine oxide (47.46 ml) was diluted to volume in a 200 ml volumetric flask with limonene, the mixture was inverted 20 times to combine the liquids. The prepared solvent was then washed with 0.1M sulphuric acid solution at a 50 ml to 50 ml ratio of solvent to wash and allowed to settle for 15 min, the heavy phase was removed and discarded.

Extraction of Morphine from the Aqueous Phase into the Organic Phase

Rich extract (20 ml, 0.025% w/v-0.0.5% w/v) was adjusted from ˜12 via addition of 5M sulphuric acid dropwise with mixing until pH 9.0 was achieved. This aqueous phase was then combined with 20 ml of the washed organic phase and mixed on a vertical orbital (Ratek suspension mixer) for 60 minutes. The biphasic system was allowed to separate and each phase was assayed. The single stage extraction efficiency for morphine was determined as 86%.

### Example 5: Extraction of Oripavine Using 0.2 M Alkyl Phosphine Oxide of Formula A in Xylene

650 mL of rich extract of oripavine was warmed to room temperature, 17-20° C. prior to the experiment. The initial pH of rich extract was recorded, then 10 w/v % NaOH was used to adjust the pH to 9.0. 180 mL of pH 9.0 rich extract was then dispensed into a 250 mL separating funnel in 3 replicates. 30 mL of 0.2 M alkyl phosphine oxide of Formula A in xylene was added to the separating funnel, then shaken vigorously 2 minutes. The separating funnel was allowed to stand for 15 minutes for phase separation. The pH of the aqueous layer was measured, H₂SO₄ (0.1 and 0.5 M) and NaOH (10% w/v) were used for pH adjustment if the pH deviates±0.15 from the target pH 9.0. The two phases were separate and were analyzed using Ultra Performance Liquid Chromatography (UPLC). The single stage extraction efficiency for oripavine was determined as 96%.

In similar experiments, the extraction yield of oripavine in 0.2 M alkyl phosphine oxide of Formula A in limonene was 69.9%, in 0.6 M alkyl phosphine oxide of Formula A in xylene the yield was 88.6% and in 0.6 M alkyl phosphine oxide of Formula A in limonene it was 88.1%. It is seen that increasing the concentration of alkyl phosphine oxide of Formula A from 0.2 M to 0.6 M improved the extraction yield for both xylene and limonene. At 0.2 M concentration, xylene showed to have higher efficiency (81.5% compared to 69.9% for Limonene). For 0.2 M alkyl phosphine oxide of Formula A in limonene, at pH 9.0, D is 14.5, which gives 93.3% of extraction efficiency in solvent phase. With aqueous:solvent ratio 180 mL: 30 mL, the theoretical number of stage for extraction is 3.7, whereas for extraction of oripavine from rich extract into xylene with D value of 24.8 corresponding to 96% of extraction efficiency in solvent phase, the theoretical number of stage for extraction is 2.48.

### Example 6: Extraction of Codeine Using 0.2 M Alkyl Phosphine Oxide of Formula A in Xylene

The initial pH of rich extract of codeine (REC) was recorded, and then was adjusted to pH 9.0 using 50% w/v H₂SO₄. REC was then dispensed into a separating funnel in 3 replicates. 0.2 M alkyl phosphine oxide of Formula A in xylene was added to the separating funnel, then shaken vigorously 2 minutes. The separating funnel was allowed to stand for 15 minutes for phase separation. The pH of the aqueous layer was measured, H₂SO₄ (0.1 and 0.5 M) and NaOH (10% w/v) were used for pH adjustment if the pH deviates ±0.15 from the target pH 9.0. The two phases were separate and were analyzed using UPLC. In a similar experiment, the extraction yield of codeine in 0.2 M alkyl phosphine oxide of Formula A in limonene was 10.3%.

1. A process for the extraction of alkaloids from a source, using an alkyl phosphine oxide as an extractant;
wherein the alkyl phosphine oxide is represented by Formula A: 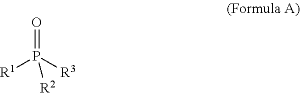
wherein R¹, R² and R³ are each independently selected from C₁-C₁₀ alkyl which is straight chain, branched or cyclic alkyl.

2. The process according to claim 1, wherein the extraction of alkaloids from the source comprises the step of contacting an aqueous solution of an alkaloid source with an organic layer consisting of an alkyl phosphine oxide of Formula A as an extractant and a diluent selected from xylene or limonene.

3. The process according to claim 1, wherein
the R¹, R² and R³ of alkyl phosphine oxide of Formula A are independently selected from hexyl or octyl; or
the extractant alkyl phosphine oxide of Formula A is selected from the group consisting of dioctyl-monohexyl phosphine oxide, monooctyl-dihexyl phosphine oxide, trioctyl phosphine oxide and trihexyl phosphine oxide, and a mixture thereof.

4. (canceled)

5. The process according to claim 1, wherein the extraction is performed at a pH ranging from 8 to 10,
at organic phase:aqueous phase ratio ≤1, or
a molar con 0.2M to 0.6M of the extractant.

6. (canceled)

7. (canceled)

8. The process according to claim 1, wherein the extraction of alkaloids from a source comprises the steps of,
(a) preparing an aqueous phase solution of alkaloids by mixing source with water;
(b) separately, preparing an organic phase consisting of an alkyl phosphine oxide of Formula A as extractant and a diluent selected from xylene or limonene;
(c) contacting the aqueous solution of step (a) containing alkaloids with an organic layer consisting of an alkyl phosphine oxide of Formula A as extractant and a diluent selected from xylene or limonene of step (b) having molar concentration of extractant from 0.2M to 0.6M;
(d) separating the organic phase; and
(e) back extracting the organic phase to provide alkaloids.

9. The process according to claim 1, wherein the alkaloids are selected from morphine, codeine, oripavine, thebaine or noscapine, or wherein the alkaloids are selected from morphine, codeine or oripavine.

10. (canceled)

11. The process according to claim 409, wherein the multi-stage extraction efficiency of morphine is >95%, or where the extraction efficiency of oripavine is >95%.

12. (canceled)

13. The process according to claim 199, wherein in step (c), the pH of the aqueous layer maintained between the range of 8 and 10,
wherein the ratio of aqueous phase to organic phase is ≤1; or
wherein the back extraction in step (e) is carried out using sodium hydroxide solution.

14. (canceled)

15. (canceled)

16. A liquid-liquid extraction process for the extraction of alkaloids from a source using an alkyl phosphine oxide of Formula A: 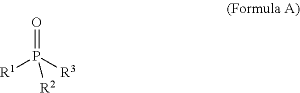
wherein R¹, R² and R³ are each independently selected from C₁-C₁₀ alkyl which is straight chain, branched or cyclic alkyl, as an extractant in combination with a diluent selected from xylene or limonene in an extraction column, wherein the said process comprises the steps of:
(i) installing an organic phase distributor at the bottom of the column, consisting an outlet port of organic phase at the top of the column, and installing an aqueous phase distributor at the top of the column, consisting an outlet port of aqueous phase at the bottom of the column,
(ii) disbursing the aqueous phase and organic phase into the column, whereby the two phases undergo extraction and mass transfer using a stack of reciprocating plates inside the column, at pH ranging from 8 to 10, and
(iii) separating the organic phase and subsequent back extraction to provide alkaloids.

17. The process according to claim 16, wherein
the extraction column is prepared as depicted in FIG.-1,
a stack of reciprocating plates is placed inside the extraction column,
the stack of reciprocating plates is made of stainless steel and placed in the centre of the column, or
the stack of plates reciprocated using a variable speed pump installed at the top of the column, and the reciprocating amplitude is adjusted through a slider crank with a fly wheel which was attached to the pump.

18. (canceled)

19. (canceled)

20. (canceled)

21. A process according to claim 1 for the extraction of an alkaloid from the source comprising use of alkyl phosphine oxide or a mixture of alkyl phosphine oxides as an extractant.

22. The process according to claim 21, wherein the alkyl phosphine oxide is represented by Formula A: 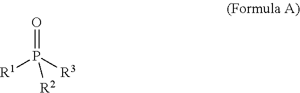
wherein R¹, R² and R³ are each independently selected from C₁-C₁₀ alkyl which is straight chain, branched or cyclic alkyl.

23. The process according to claim 22, wherein the alkyl phosphine oxide of Formula A is selected from the group consisting of dioctyl-monohexyl phosphine oxide, monooctyl-dihexyl phosphine oxide, trioctyl phosphine oxide and trihexyl phosphine oxide, and a mixture thereof.

24. The process according to claim 21, wherein the alkyl phosphine oxide is diluted using diluent selected from xylene or limonene, and wherein the concentration of the alkyl phosphine oxide in diluent is from 0.2 M to 0.6 M.

25. An alkaloid or alkaloids obtained by the process according to claim 1.

26. An alkaloid or alkaloids according to claim 25, wherein the alkaloid or alkaloids are selected from morphine, codeine, oripavine, thebaine or noscapine, or wherein the alkaloid or alkaloids are subsequently converted to a therapeutically effective compound or derivatives thereof.

27. (canceled)

28. The alkaloid or alkaloids according to claim 25, which are subsequently converted to a therapeutically effective compound or derivatives thereof, selected from naltrexone, nalbuphine and buprenorphine.

29. (canceled)

30. (canceled)

31. A process according to claim 1 for the extraction of morphine, codeine or oripavine from the source, wherein the process comprises use of an extractant selected from the group consisting of dioctyl-monohexyl phosphine oxide, monooctyl-dihexyl phosphine oxide, trioctyl phosphine oxide and trihexyl phosphine oxide, and a mixture thereof.

32. The process according to claim 31, wherein
the extractant is a composite mixture comprising dioctyl-monohexyl phosphine oxide (10-22%), monooctyl-dihexyl phosphine oxide (10-16%), trioctyl phosphine oxide (5-8%) and trihexyl phosphine oxide (5-8%),
the extractant is diluted is xylene or limonene and the molar concentration of the extractant in the diluent is from 0.2 M to 0.6 M,
the single-stage extraction efficiency of morphine is not less than 65%, or
the single-stage extraction efficiency of oripavine is not less than 90%.

33. (canceled)

34. (canceled)

35. (canceled)

36. A process for the extraction of alkaloid from the source, using an alkyl phosphine oxide or mixtures thereof as an extractant; wherein the alkyl phosphine oxide is represented by Formula A: 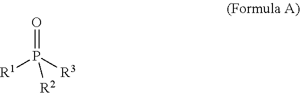
wherein R¹, R² and R³ are each independently selected from hexyl or octyl, and wherein the extraction process comprises:
a. preparing an aqueous phase solution of alkaloids by mixing the source with water;
b. separately, preparing an organic phase consisting of the extractant;
c. contacting the aqueous solution of step (a) with the organic phase of step (b); and
d. separating the organic phase.

37. The process according to claim 36, wherein
the extractant is selected form the group consisting of dioctyl-monohexyl phosphine oxide, monooctyl-dihexyl phosphine oxide, trioctyl phosphine oxide and trihexyl phosphine oxide, and a mixture thereof,
the extractant is a composite mixture comprising dioctyl-monohexyl phosphine oxide (10-22%), monooctyl-dihexyl phosphine oxide (10-16%), trioctyl phosphine oxide (5-8%) and trihexyl phosphine oxide (5-8%), or
the organic phase in step (b) is prepared by diluting the extractant in dilutent selected from xylene or limonene.

38. (canceled)

39. (canceled)

40. The process according to claim 37, wherein the molar concentration of the extractant is from 0.2 M to 0.6 M.

41. The process according to claim 36, wherein
the pH of the aqueous layer in step (c) is between 8 and 10,
the ratio of volume of aqueous to organic layer is less than or equal to 1,
the organic phase separated in step (d) is back extraction out using sodium hydroxide solution, or
the alkaloid is morphine, codeine, oripavine, thebaine or noscapine.

42.-47. (canceled)
